(19) United States
(12) Patent Application Publication
(10) Pub. No.: US 2013/0017218 A1
(43) Pub. Date: Jan. 17, 2013

(54) LIVE BACTERIAL VACCINE SAFETY

(71) Applicant: Matthew K. Waldor, Newton, MA (US)

(72) Inventor: Matthew K. Waldor, Newton, MA (US)

(73) Assignee: THE BRIGHAM AND WOMEN'S HOSPITAL, INC., Boston, MA (US)

(21) Appl. No.: 13/577,667

(22) PCT Filed: Feb. 11, 2011
(86) PCT No.: PCT/US2011/024429

§ 371 Date: Oct. 3, 2012

Related U.S. Application Data
(60) Provisional application No. 61/303,804, filed on Feb. 12, 2010.

(51) Int. Cl. A61K 39/02; A61P 37/04; A61K 39/112; A61K 39/104; A61K 39/106; A61K 39/108

(52) U.S. Cl. 424/235.1

(57) ABSTRACT

The present invention provides for a method of reducing the reactogenicity of live attentuated bacterial vaccines by deleting at least a portion (e.g., the TLR-5 stimulating domain of a flagellin protein) of at least one of the bacterial genes that encodes a flagellin from the bacterium genome. These vaccines are directed against enteric or non-enteric flagellin-producing bacteria. A particular embodiment provides for a live attenuated cholera vaccine having reduced reactogenicity.

### CROSS REFERENCE TO RELATED APPLICATIONS

This application claims the benefit of priority under 35 U.S.C. §119(e) of the U.S. Provisional Application No. 61/303,804 filed Feb. 12, 2010, the contents of which are incorporated herein by reference in its entirety.

### FEDERAL FUNDING LEGEND

This invention was made with government support under Grant No. R37-AI-42347 and Grant No. UL1 RR 025758-02 awarded by the National Institutes of Health. The U.S. government has certain rights in the invention.

### FIELD OF THE INVENTION

The present invention provides for methods of enhancing the safety of live attenuated bacterial vaccines. More specifically, the safety of live attenuated bacterial vaccines is improved by removal of the flagellin proteins. This approach can be applied to a variety of live vaccines that are based on bacteria that produce flagellins.

### BACKGROUND

Many live attenuated bacterial vaccines are being created or are in clinical trials. For example live vaccines are being developed for cholera, a disease caused by Vibrio cholerae, a motile gram-negative bacterium. Even when the genes for the major toxin of this pathogen are deleted, human trials have shown that live attenuated vaccines cause residual diarrhea or other symptoms of reactogenicity. Hence, there is a need for live attenuated bacterial vaccines with improved safety.

### SUMMARY

An object of the present invention provides for live attenuated bacterial vaccines with improved safety. More specifically, the present invention shows that residual reactogenicity of live attenuated vaccines, often characterized by diarrhea, is attributable to flagellin proteins. These vaccines are directed against enteric or non-enteric flagellin-producing bacteria. For example, the deletion of the genes coding for the V. cholerae flagellin proteins abolished the diarrhea associated with administration of live vaccine constructs. Furthermore, because flagellin proteins may stimulate diarrhea by activating the TLR-5 pathway of innate immunity, live attenuated vaccines may be improved by deleting only the TLR-5 stimulating domain of flagellin proteins.

The present invention provides for non-reactogenic bacterial vaccine strains that do not elicit diarrhea in an infant rabbit model of disease. These vaccines are suitable for agriculturally relevant animals, and humans. Bacterial disease agents included in the embodiments of the present invention include flagellin-producing bacteria such as Vibrio, Escherichia coli, Campylobacter, Salmonella, Shigella, Aeromonas, or Pseudomonas. More specifically, bacteria for which vaccine reactogenicity may be reduced include Vibrio cholerae, pathogenic Escherichia coli, (i.e., enterotoxigenic E. coli, enterohemmorrhagic E. coli, enteroaggregative E. coli, and enteroinvasive E. coli), Campylobacter jejuni, Vibrio parahemolyticus, Salmonella enterica and other Salmonella spp., Shigella spp., Aeromonas hydrophila, or other flagellin-producing bacteria including Pseudomonas aeroguniosa and extraintestinal E. coli such as uropathogenic E. coli.

### DESCRIPTION OF THE DRAWINGS

FIG. 1. Diarrhea in infant rabbits inoculated with Peru-NT (a ctxAB mutant) or one of its derivatives. Rabbits exhibiting severe, mild and no diarrhea are shown in 1A, 1B and 1C, respectively. The frequency with which rabbits of various genotypes exhibited diarrhea, as well as statistical analyses of these results, are presented in 1D.

FIG. 2. Motility, flagellum and flagellin production in Peru-NT or one of its derivatives. (2A) The indicated strains were inoculated into 0.3% LB agar and photographed 12 h later. Peru-NT (2B) or Peru-NTΔflaABCDE (2C) were visualized using transmission electron microscopy. Scale bar equals 1 μm. (2D) A Western blot of whole cell extracts from the indicated strains was probed with 1:4000 dilution of antisera to V. parahaemolyticus polar flagellins. Scott, 98 PNAS 13978-83 (2001).

FIG. 3. Intestinal colonization of Peru-NT or one of its derivatives. The number of CFU in tissue homogenates of the proximal (3A), mid (3B) and distal (3C) small intestine (SI) and mid-colon (3D) 3 days after inoculation are shown in each graph. The bar shows the geometric mean for each group.

FIG. 4. Relative levels of transcripts for proinflammatory cytokines in homogenates from the small (4A) and large (4B) intestines of infant rabbits inoculated with Peru-NT (gray bars) or Peru-NTΔflaABCDE (black bars). Homogenates were obtained 3 days post-inoculation. Transcript levels were determined by quantitative real-time PCR and normalized to GAPDH cDNA levels. The results are shown as log₂ difference relative to the levels measured in samples from control rabbits inoculated with buffer. The stars indicate statistically significant (P<0.05) different values in Peru-NT and Peru-NTΔflaABCDE samples.

FIG. 5. Electron micrographs showing the presence or absence of flagella for Peru-NT and its derivatives (5A) Peru-NT; (5B) Peru-NTΔflaABCDE; (5C) Peru-NTΔmotB; (5D) Peru-NTΔflaA; (5E) Peru-NTΔflaACD; (5F) Peru-NTΔflaBCDE; and (5G) Peru-NTΔflaABCDE pSW-flaA.

### DETAILED DESCRIPTION

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Bacterial diseases such as cholera have afflicted humans for thousands of years, and remain a significant threat to health in many parts of the developing world, especially in Africa and Asia. For example, cholera is a severe diarrheal disease caused by the motile gram-negative rod Vibrio cholerae. There are several million cases of cholera in the world annually (WHO, Cholera unveiled (2003)), and that more than 100,000 people per year die from this infection. Although re-hydration therapy is effective and greatly reduces mortality when available, the continued burden of cholera, particularly in regions with socio-economic disruptions, has prompted the recommendation that vaccines to protect against infection with El Tor biotype V. cholerae, the cause of the ongoing 7th pandemic, be developed. Chaignat & Monti, 25 J. Health Popul. Nutr. 244-61 (2007); WHO, 76 Wkly Epidemiol. Rec. 117-24 (2001).

Attenuation refers to the production of strains of pathogenic microorganisms that have essentially lost their disease-producing ability. Mutants are selected which have lost virulence but remain capable of eliciting an immune response. Attenuated pathogens often make good immunogens as they replicate in the host cell and elicit long lasting immunity. Several problems are encountered with the use of live vaccines, however, such as insufficient attenuation and the risk of reversion to virulence, but side effects from reactogenic vaccines also remain an ongoing concern in vaccine safety.

Live attenuated V. cholerae vaccines harboring deletions of the genes encoding cholera toxin have great promise for reducing the global burden of cholera. Implementation of live vaccines has been hampered, however, by the tendency of such strains to induce non-choleric “reactogenic” diarrhea. Previously, the molecular bases of reactogenicity were unknown. The present inventor discovered that reactogenic diarrhea is a response to V. cholerae's flagellar proteins. An infant rabbit model of reactogenicity showed which V. cholerae factors trigger this response: V. cholerae ctx mutants that produced flagellins induced diarrhea, whether or not the proteins were assembled into a functional flagellum. In contrast, ˜90% of rabbits infected with V. cholerae that lacked all five flagellin-encoding genes did not develop diarrhea. Thus, flagellin production, independent of flagellum assembly or motility, is sufficient for reactogenicity. The intestinal colonization and intra-intestinal localization of a non-reactogenic flagellin-deficient strain were indistinguishable from those of a flagellated motile strain; however, the flagellin-deficient strain stimulated less production of mRNA transcripts coding for pro-inflammatory cytokines in the intestine. Thus, reactogenic diarrhea may be a consequence of an innate host inflammatory response to V. cholerae flagellin proteins. This invention thus provides for a simple genetic blueprint for the engineering of defined non-reactogenic live-attenuated bacterial, e.g., V. cholerae, vaccine strains.

V. cholerae is a non-invasive enteric pathogen, contracted by ingesting contaminated water or food. Bacteria that survive passage through the acidic gastric barrier colonize the small bowel, where they produce cholera toxin (CT), an A-B₅-subunit type exotoxin. CT is thought to be the principal factor underlying the severe secretory diarrhea that is characteristic of cholera. Sanchez & Holmgren, 65 Cell Mol. Life. Sci. 1347-60 (2001). In support of this idea, human volunteers who ingested CT developed cholera-like diarrhea. Levine et al., 47 Microbiol. REv. 510-50 (1983).

Since the initial cloning of ctxAB, the genes encoding the A and B subunits of CT (Mekalanos et al., 306 Nature 551-57 (1983)), there have been several attempts to engineer live-attenuated V. cholerae vaccine strains via deletion of ctxA, which encodes the toxic moiety of CT. Ryan et al., 5 Expert Rev. Vaccines 483-94 (2006). To date, ctxA live-attenuated oral V. cholerae vaccine strains have shown promise, but many of the candidate vaccine strains have led to side effects in the human volunteers who ingested them. Such side effects, often referred to as vaccine ‘reactogenicity’, include non-choleric diarrhea and abdominal cramps. Id. Comparative analyses of vaccine candidates suggests that reactogenicity may be linked to V. cholerae's single polar flagellum and/or to bacterial motility. The vaccine strain Peru-3, a ctxA derivative of a Peruvian El Tor clinical isolate, caused diarrhea, whereas Peru-15, a spontaneously derived non-flagelated (non-motile) derivative of Peru-3, did not. Taylor et al., 170 J. Infect. Dis. 1518-23 (1994); Kenner et al., 172 J. Infect. Dis. 1126-29 (1995). Both strains engendered protection against challenge with wild type V. cholerae in human trials, suggesting that the lack of reactogenicity is not simply due to a failure of Peru-15 to colonize. Despite these initial findings, however, the precise cause of reactogenic diarrhea has not been identified, and the nature of the genetic changes that distinguish Peru-15 from Peru-3 remain unknown.

Several (not necessarily exclusive) hypotheses regarding the origins of reactogenicity have been proposed. For example, it is possible that flagellation and motility enable V. cholerae to penetrate the mucus layer covering the intestinal epithelial surface, and that the close proximity of the organism to the apical surface of epithelial cells elicits an inflammatory response that results in diarrhea. Mekalanos & Sadoff, 265 Science 1387-89 (1994); Mekalanos et al., 93 Bull. Inst. Pasteur 255-62 (1995). Additionally, it is possible that reactogenicity is induced by toxins still produced by the ctxA mutant, such as zonula occludens toxin, accessory cholera enterotoxin, hemolysin A, MARTX toxin, and/or hemagglutinin/protease, through direct enterotoxicity and/or through pro-inflammatory effects. Fasano et al., 88 PNAS 5242-46 (1991); Satchell, 5 Microbes Infect. 1241-47 (2003). The effect of these factors might be potentiated by close contact between the bacteria and the epithelium. Further, recent work using tissue culture models has led to the hypothesis that the 5 V. cholerae flagellins, which have been demonstrated to activate the Toll-like receptor 5 (TLR5) signaling pathway, could lead directly to reactogenic diarrhea by stimulating production of pro-inflammatory cytokines in the intestine. Harrison et al., 76 Infect. Immun. 5524-34 (2008); Xicohtencatl-Cortes et al., 5 Mol. Cell. Proteomics 2374-84 (2006). Detection of lactoferrin and fecal leukocytes in the stools of volunteers with reactogenic diarrhea supports the idea that intestinal inflammation is associated with vaccine reactogencity. Silva et al., 64 Infect. Immun. 2361-64 (1996); Qadri to cl., 53 Gut 62-69 (2004).

Investigation of the molecular basis of V. cholerae vaccine reactogenicity has been hampered by the lack of an animal model. Recently, it was found that infant rabbits can serve as a model for severe cholera as well as for the reactogenic diarrhea caused by V. cholerae ctxA mutants (Ritchie et al., submitted). Oro-gastric inoculation of wild type V. cholerae into infant rabbits that had been pre-treated with cimetidine led to lethal, watery diarrhea in virtually all animals. Rabbits inoculated with wild type V. cholerae usually died about 24 to 30 hours later. In contrast, rabbits inoculated with an isogenic V. cholerae ctxAB mutant exhibited no or minimal signs of disease during this time period; yet about 36 to 60 hours after inoculation of the ctxAB mutant, more than 90% of the animals developed non-choleric ‘fecal diarrhea’ that resolved within about 24 hours.

The present invention used infant rabbits to explore the genetic basis of reactogenic diarrhea. The reactogenic vaccine V. cholerae strain Peru-3 caused diarrhea in most rabbits, whereas the non-reactogenic strain Peru-15 did not, thereby validating the relevance of the rabbit model for study of reactogenicity. Subsequently, an isogenic set of defined mutants showed whether motility per se, production of a flagellum, or production of flagellin proteins underlies reactogenicity. These experiments revealed that neither motility nor a flagellum was required to induce reactogenic diarrhea; instead, production of flagellin proteins was sufficient cause. The intestinal colonization and the intra-intestinal localization of the non-reactogenic flagellin-deficient strain were indistinguishable from those of a flagellated motile strain; but the flagellin-deficient strain stimulated less mRNA transcripts coding for the pro-inflammatory cytokines IL-8, IL-1β and TNFα in the intestine. These data are consistent with the possibility that reactogenic diarrhea is linked to an innate host inflammatory response to V. cholerae flagellins, and they suggest a simple genetic blueprint for creation of defined non-reactogenic live-attenuated V. cholerae vaccine strains.

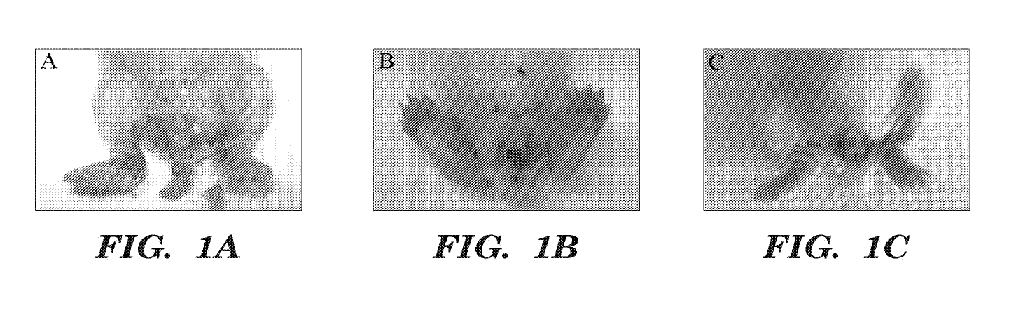

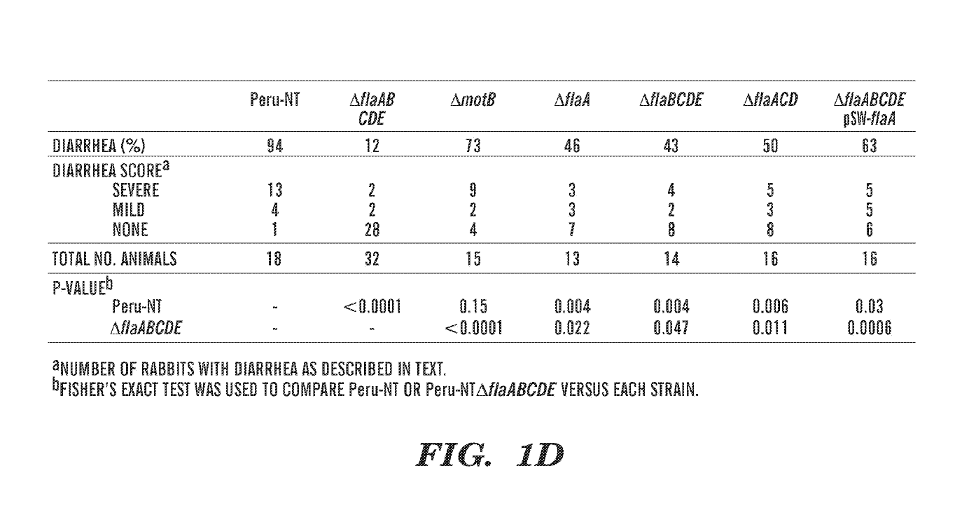

Hence, the present invention provides for non-reactogenic bacterial vaccine strains do not elicit diarrhea in an infant rabbit model of disease. In recent work developing an infant rabbit model of cholera pathogenesis, infant rabbits infected with V. cholerae lacking ctxAB developed non-choleric diarrhea, and proposed that this animal model might also be useful for study of vaccine reactogenicity (Ritchie et al., submitted). Animals infected with a C6706 ctxAB mutant (here termed Peru-NT), a derivative of a 1991 El Tor Peruvian clinical isolate (Dziejman et al., 99 PNAS 1556-61 (2002)), were contaminated with loosely adherent fecal material on their perineums, hind legs, and tails by about 36 to 60 hours after inoculation (FIG. 1). At necropsy, the large intestine contained soft, unformed fecal material; in contrast, the intestines of mock-infected rabbits contained hard, formed pellets. This “fecal” diarrhea appeared markedly different from the watery, mucin-rich fluid released from rabbits infected with wild-type V. cholerae, which closely approximates the “rice-water stool” produced by cholera patients. Fecal diarrhea was subjectively classified as severe (e.g. FIG. 1A) or mild (e.g., FIG. 1B) based upon the amount of adherent feces. Almost all (thirteen of eighteen) rabbits infected with Peru-NT developed severe diarrhea, and only one remained clear of any fecal contamination. Diarrhea spontaneously remitted by about 80 hours after inoculation. In control experiments, we found that no rabbits (none of thirteen) inoculated with buffer alone developed diarrhea.

The relevance of this animal model for studies of vaccine reactogenicity was confirmed by comparing the signs of disease exhibited by infant rabbits inoculated with either Peru-3 or Peru-15, two live-attenuated ctxA mutant vaccine strains that have been tested in humans. Volunteers inoculated with Peru-3 often developed self-limiting diarrhea, whereas diarrhea was not observed in volunteers who received Peru-15. Taylor et al., 1994; Kenner et al., 1995. Similarly, most (twelve of eighteen) rabbits inoculated with Peru-3 developed diarrhea, while only two of thirteen rabbits inoculated with Peru-15 exhibited diarrhea. These observations suggest that infant rabbits are a valid model host for study of reactogenic diarrhea caused by ctx mutant live-attenuated V. cholerae vaccine strains.

Reactogenicity depends on flagellin proteins, but not motility. Peru-15 was isolated as a spontaneous non-motile derivative of Peru-3 (Kenner et al., 1995), and the mutation(s) that render Peru-15 non-flagellated (and hence non-motile) are not known. In principle, the difference between the reactogenicity of Peru-3 and Peru-15 could result from Peru-15's lack of a flagellum and/or flagellar proteins, from the strain's lack of motility, or even from a mutation not linked to flagellation/motility. Furthermore, the differences between these strains might not have a direct connection to diarrheagenic pathways, but might instead be indirectly coupled, for example, via differences in their capacities to colonize the rabbit intestinal tract. Thus, derivatives of Peru-NT, containing a variety of mutations within genes needed for flagellar assembly and/or activity, were generated. Flagellar synthesis is a complex process that is coordinately regulated with several virulence-linked pathways (Syed et al., 191 J. Bacterial. 6555-70 (2009)); but the mutations generated for the present invention disrupted only the synthesis of the individual flagellins, and are not expected to influence other processes.

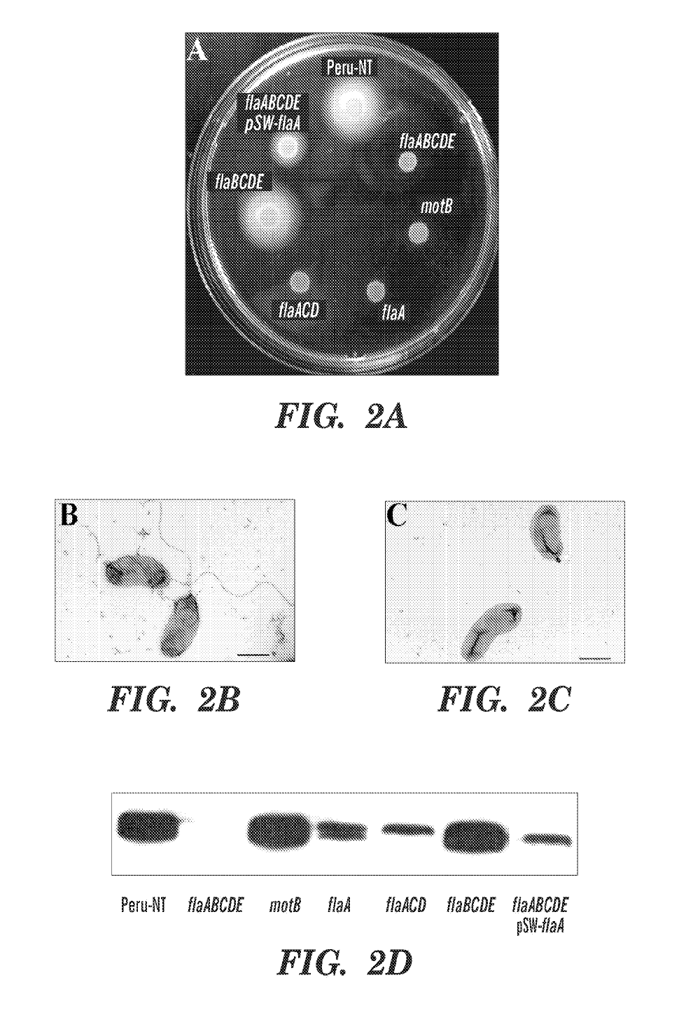

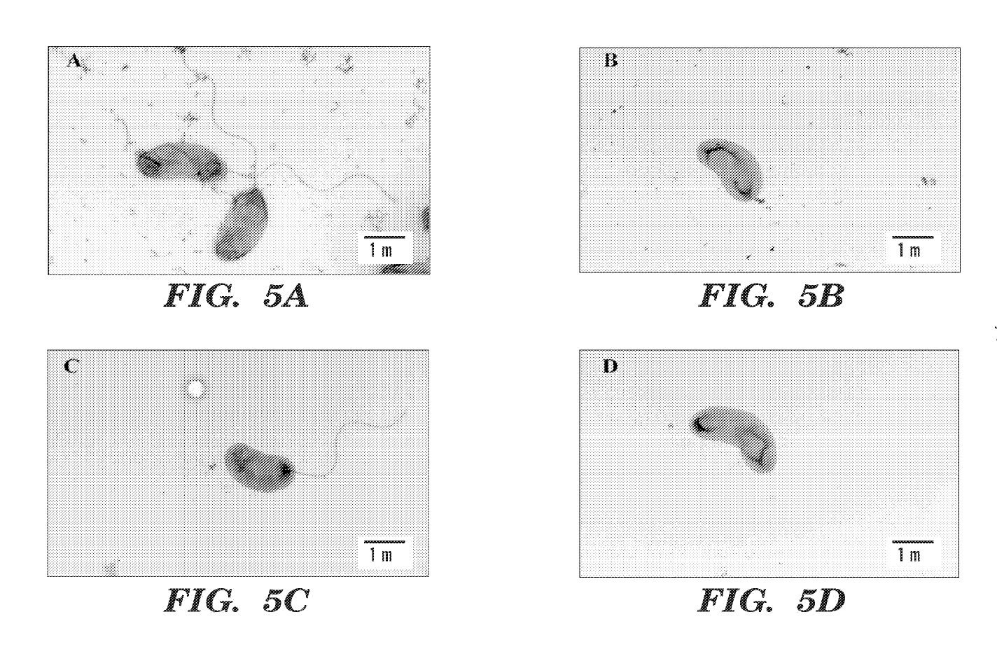

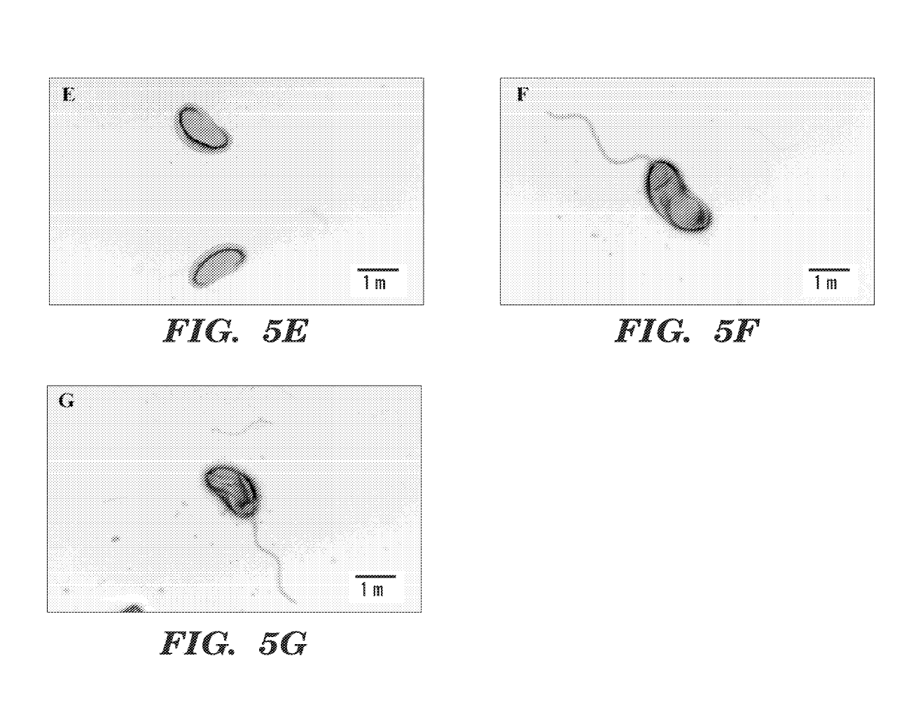

V. cholerae encodes five distinct flagellins within two operons: flaAC and flaDBE. Klose & Mekalanos, 180 J. Bacteriol. 303-16 (1998). All of these flagellins are thought to be incorporated into V. cholerae's single polar flagellum (visible in FIG. 2B); however, incorporation is dependent upon flaA. In the absence of flaA, the synthesis of other flagellins does not appear to be altered, but these flagellins are secreted, rather than incorporated into a filament. Harrison et al., 76 Infect. Immun. 5524-34 (2008). Derivatives of Peru-NT lacking 1 or more flagellin-encoding gene(s) were generated. Mutants lacking flagellins other than flaA were flagellated and motile, as expected, while strains lacking flaA were non-flagellated and non-motile (FIG. 2A and FIG. 5). The relative levels of flagellins produced by these strains are shown in FIG. 2C. As expected, Peru-NTΔflaABCDE, a strain deleted for all five V. cholerae flagellins, did not produce detectable flagellins (FIG. 2D). A strain lacking motB, which encodes a component of the flagellar motor, was also generated. This mutant synthesizes wild-type levels of flagellins and assembles a flagellum (see FIG. 2D and FIG. 5); however, this flagellum does not turn and consequently the bacteria are non-motile (FIG. 2A). See Gardel & Mekalanos, 64 Infect. Immun. 2264-55 (1996). None of the mutants used in this study displayed any significant defects in growth in vitro.

The diarrhea caused by Peru-NT, Peru-NTΔflaABCDE, and Peru-NT motB was compared after their oro-gastric inoculation into infant rabbits. The majority (73%) of rabbits infected with the motB mutant developed diarrhea, a frequency that did not differ significantly from that observed with Peru-NT (94%). In marked contrast, PeruNTΔflaABCDE led to diarrhea in only 12% (four of thirty-two) of inoculated rabbits (FIG. 1D; P<0.0001 relative to Peru-NT). Together, these data are consistent with the possibility that a functional flagellum (i.e., motility) is not required to induce reactogenicity, but that production of flagellin proteins and/or a flagellar structure are critical.

To assess whether the flagellum itself is required for diarrhea, and to begin to decipher which flagellin(s) promote reactogenic diarrhea, rabbits were infected with derivatives of Peru-NT that lacked a subset of V. cholerae's flagellin-encoding genes. Two non-flagellated strains, Peru-NTΔflaA and Peru-NTΔflaACD (FIG. 5), also caused diarrhea (FIG. 1D), albeit at a significantly lower frequency than Peru-NT (P<0.01 for both strains versus Peru-NT). Therefore, the flagellum filament is not essential for diarrhea. Because flagellins are thought to be secreted in the absence of flagellum production, these observations suggest that extracellular flagellin monomers can induce reactogenic diarrhea. Furthermore, diarrhea does not appear to be linked to a particular flagellin monomer. As noted above, strains lacking flaA caused diarrhea; additionally, rabbits inoculated with Peru-NTΔflaBCDE and Peru-NTΔflaABCDE pSW-flaA (which expresses flaA from a low copy vector) developed diarrhea. Thus, no individual flagellin is essential for induction of diarrhea, although flaA appears to be sufficient. Notably, all of the strains lacking at least one but not all flagellin-encoding gene(s) differed significantly from both Peru-NT and Peru-NΔflaABCDE in their frequency of causing diarrhea. This result suggests that although a subset of flagellins can be sufficient to induce reactogenic diarrhea, the full complement of flagellins is a more potent stimulus. There does not appear to be a precise correlation between reactogenicity and net production of flagellin monomers (compare FIGS. 1D and 2C); however, it is quite possible that the abundance of cell-associated monomers does not accurately reflect the level of secreted monomers.

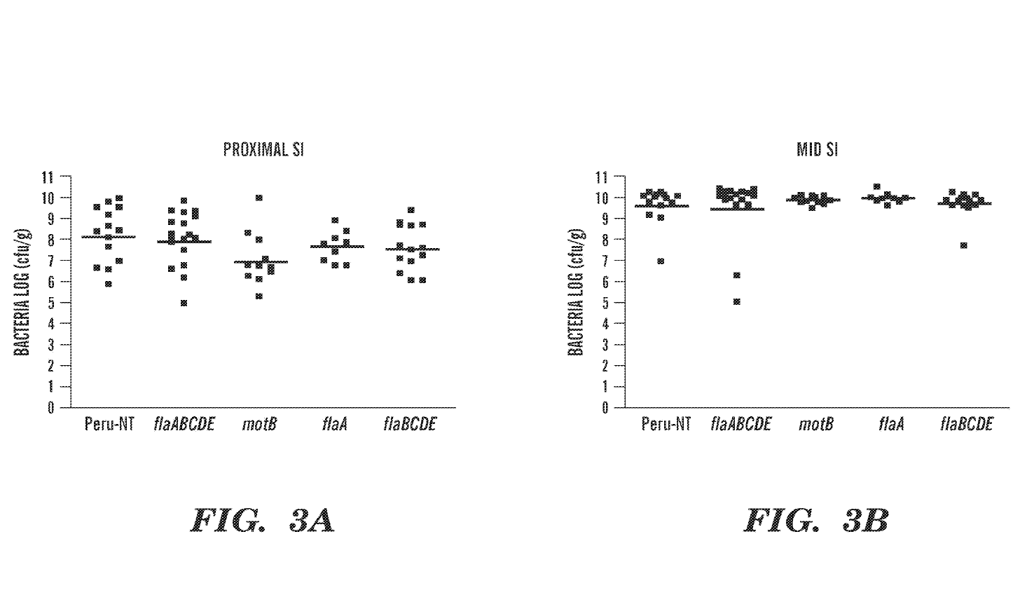

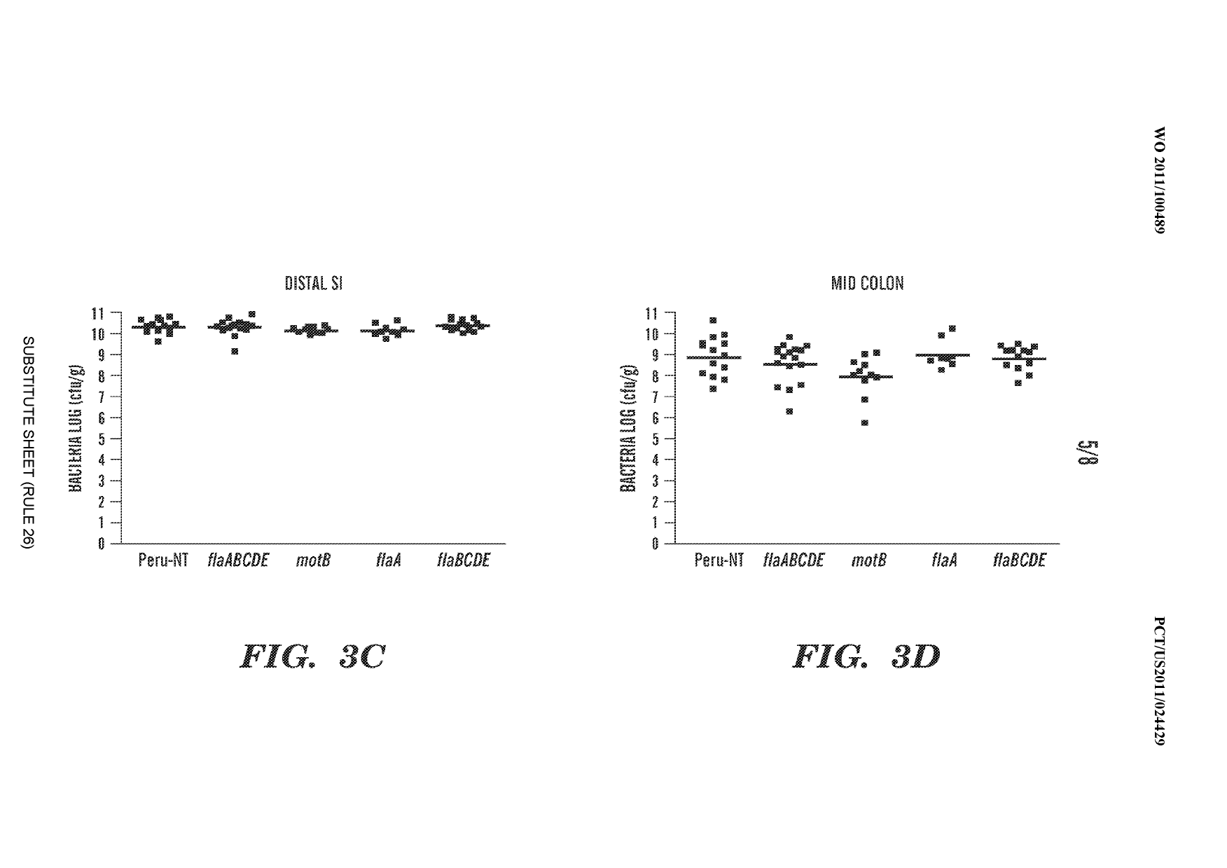

Differential intestinal colonization or localization of Peru-NT flagellin mutants does not account for reactogenic diarrhea. It is possible that the results presented above do not indicate a direct reactogenic role for flagellin proteins, but instead reflect differences among the colonization capacities of the various strains. To explore this possibility, we determined the number of colony forming units (CFU) recovered for each strain in intestinal tissue homogenates three days after their inoculation into infant rabbits. All strains robustly colonized the mid- and distal portions of the small intestine (˜10¹⁰ CFU g⁻¹) as well as the mid colon. The number of CFU recovered from animals infected with the flagellin mutants did not differ significantly from the number of Peru-NT CFU recovered at any site (FIG. 3). These findings argue strongly against the idea that the capacity of these strains to colonize the intestine, at least as assessed by CFU recovered in intestinal homogenates, correlates with their stimulation of diarrhea.

Although the numbers of Peru-NT and Peru-NTΔflaABCDE CFU recovered from the intestine were similar, the fine localization of these strains within the intestine could differ, given their dramatic differences in motility. To address this issue, Peru-NT and Peru-NTΔflaABCDE within infected tissues were visualized using confocal microscopy. Unexpectedly, we observed that these strains exhibited very similar patterns of localization in the small and large intestine. Like Peru-NT, the non-motile Peru-NT flaABCDE could be found in close apposition to all parts of the villous surface as well deep in the crypt-like structures of the infant rabbit intestine (data not shown). This observation strongly suggests that flagellar-based motility is not required for V. cholerae to gain access to the intestinal crypts or to get close to the epithelial surface in this model host. In the large intestine, both strains were usually found in the lumen frequently covering the surface of digesta, and occasionally in close proximity to the colonic epithelium (data not shown).

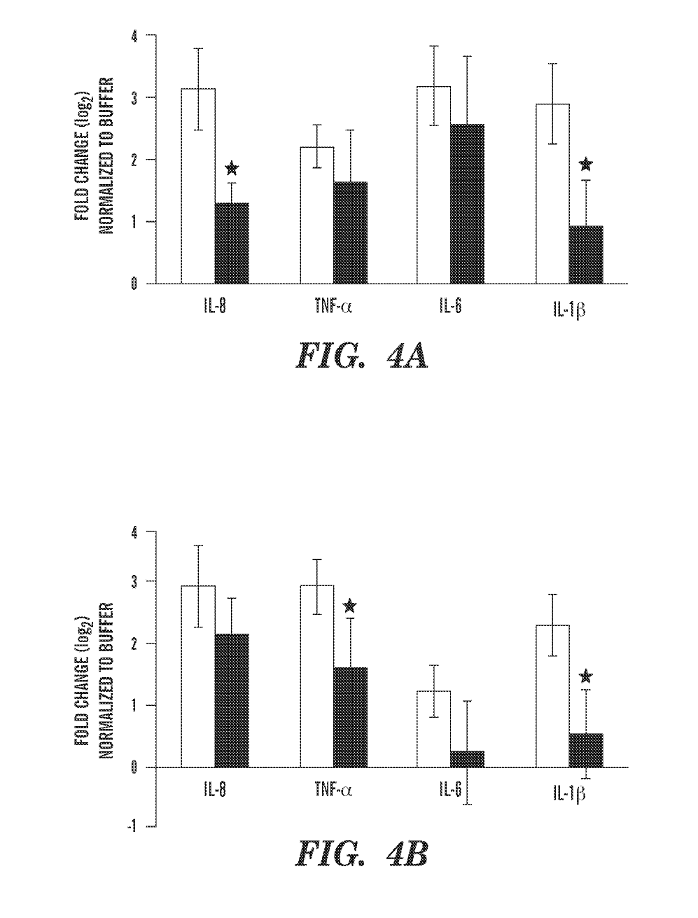

Peru-NT and Peru-NTΔflaABCDE differ in their stimulation of pro-inflammatory cytokines. Bacterial flagellin proteins, including all five V. cholerae flagellins, are known to stimulate production of proinflammatory cytokines by activation of Toll-like receptor 5 (TLR5). Harrison et al., 2008; Xicohtencatl-Cortes, 2006; Yoon & Mkalanos, 76 Infect. Immun. 1282-88 (2008). Therefore, the relative abundance of transcripts for several cytokines in tissue homogenates from rabbits infected with Peru-NT or Peru-NTΔflaABCDE were compared with those in mock-infected rabbits. Both strains led to elevations in transcripts for all cytokines measured compared to mock-infected rabbits (FIG. 4). There were ˜4 fold lower amounts of IL-8 and IL-1β transcripts, however, in tissue homogenates from the distal small intestines of rabbits infected with Peru-NTΔflaABCDE compared with those from rabbits infected with Peru-NT (FIG. 4A). Similarly, there were significantly fewer TNF-α and IL-1β transcripts in tissue samples from the mid-colons of Peru-NTΔflaABCDE infected rabbits than in Peru-NT-infected animals (FIG. 4B). Histologic examination of tissue sections from infected rabbits revealed that there were few to no heterophils (the rabbit equivalent of neutrophils) in samples from the small intestine; however, moderate numbers of heterophils were seen in the lamina propria, crossing the epithelium and amidst the digesta of the mid colon. There appeared to be more heterophils in colonic samples from Peru-NT-compared to Peru-NTΔflaABCDE-infected rabbits, although this trend did not reach statistical significance.

Live-attenuated V. cholerae vaccines have great promise for reducing the global burden of cholera, since a single oral dose often engenders long-lived protective immunity. Ryan et al., 5 Expert Rev. Vaccines 483-94 (2006). Widespread acceptance and utilization of such vaccines has been hampered, however, by their reactogenicity. The molecular bases of reactogenicity are not known, but it has been speculated, based on the absence of reactogenic diarrhea associated with Peru-15, a non-flagellated V. cholerae ctxA mutant, that symptoms are a response to V. cholerae's flagellum and/or the motility that it enables. An infant rabbit model of reactogenicity was used herein to better define the V. cholerae factors that contribute to this problem. In this model, V. cholerae ctx mutants that produced flagellins induced diarrhea, regardless of whether the proteins were assembled into a flagellum or whether the flagellum was functional. In contrast, this response was absent in ˜90% of rabbits infected with V. cholerae lacking all five flagellin-encoding genes. Thus, flagellin protein production, independent of motility or intact flagellum, is sufficient for reactogenicity.

Previous studies have demonstrated that flagellins have proinflammatory effects that can contribute to diarrhea. Flagellins have been found to interact with TLR5 and to trigger MyD88 and NF-κB-dependent transcription of proinflammatory cytokines. Harrison et al., 2008. Notably, all five V. cholerae flagellins, which can be secreted independently of flagellum filament assembly, contain the amino acid motif that stimulates TLR5, and purified V. cholerae flagellins were found to elicit TLR5-dependent IL-8 secretion from T84 cells. Id. In addition, flagellins have been shown to activate the NLRC4-inflammasome, promoting IL-1β maturation and secretion. Miao et al., 29 Semin. Immunopathol. 275-88 (2007). Release of IL-1β and other pro-inflammatory cytokines can induce diarrhea via several processes. For example, TNFα induces contraction of the actomyosin ring that controls tight junctions, resulting in diminished epithelial barrier function. Viswanathan et al., 7 Nat. Rev. Micro. 110-19 (2009); Turner, 169 Am. J. Pathol. 1901-19 (2006); Clayburgh et al., 115 J. Clin. Invest. 2702-15 (2005). In addition, IL-8 is chemotactic and promotes an influx of inflammatory cells. Neutrophil-derived 5′-AMP can lead to diarrhea by promoting Cl-secretion. Viswanathan et al., 2009. Collectively, these factors disrupt the equilibrium between the typical absorptive and secretory functions of the intestinal epithelium.

Consistent with the model outlined herein, flagellin-dependent increases in transcripts for several proinflammatory cytokines were found within tissue samples from infected rabbits. Transcripts for IL-8 and IL-1β were increased ˜8-fold in tissue samples from the small intestines of rabbits infected with Peru-NT, while their abundance in tissue from the small intestines of rabbits infected with Peru-NTΔflaABCDE differed by only ˜2-fold from those of mock-infected rabbits. Statistically significant differences between the induction of TNFα and IL-1β in tissue samples from the large intestines of Peru-NT and Peru-NTΔflaABCDE-infected rabbits were also detected. These data are consistent with the hypothesis that flagellins are released in the rabbit intestine and induce synthesis of cytokines. If this process is dependent upon TLR5, however, it is unclear how the flagellins reach TLR5, which is apparently found on the basolateral membrane of intestinal epithelial cells. Rhee et al., 102 PNAS 13610-15 (2005). It is also not currently known which site (small versus large intestine) is the primary source of reactogenic diarrhea. Histologic analyses of the small intestine did not reveal an inflammatory response, while mild to moderate inflammation was detected within the large intestines both of Peru-NT and Peru-NTΔflaABCDE-infected rabbits. The latter finding may reflect the fact that IL-8 transcripts were elevated in this tissue in response to both bacterial strains. Collectively, these data suggest that an inflammatory infiltrate (i.e., heterophils) is not sufficient to induce diarrhea, although it may be a contributing factor. Perhaps the effects of such cells must be coupled to additional factors, such as the relative elevation of transcripts for cytokines that do not act as chemoattractants. Alternatively, it is possible that flagellins prompt reactogenicity through processes in addition to, or independent of, proinflammatory cytokines.

The present work provides evidence against the idea that additional V. cholerae toxins, such as hemolysin A, MARTX toxin, or hemagglutinin/protease, are major contributors to reactogenicity. It has been proposed that these three toxins contribute to reactogenicity by promoting inflammation, primarily based on studies using a lung infection model. Fullner et al., 195 J. Exp. Med. 1455-62 (2002). All of these factors were intact in Peru-NTΔflaABCDE, however, which caused diarrhea in only 12% of rabbits. Furthermore, ten or fourteen (71%) rabbits inoculated with a Peru-NT derivative deleted for hlyA, hap, and rtx still developed diarrhea, again suggesting that these toxins are not the principal factors underlying reactogenicity. It is possible that these factors contribute to the diarrhea observed in the small minority of rabbits inoculated with Peru-NTΔflaABCDE.

The present work also argues against the idea that intestinal colonization per se leads to diarrhea, i.e., against the possibility that reactogenicity is largely a reflection of bacterial fitness within the intestine environment. No correlation was observed between the presence or absence of diarrhea in infected rabbits and the extent of colonization. In fact, there was no detectable difference between the number of CFU of Peru-NT and Peru-NTΔflaABCDE recovered from intestinal homogenates at three days post-infection. Furthermore, colonization of the large and small intestine by Peru-NT continued at a constant or even increasing level by day-six post-inoculation, even though diarrhea had ceased by this point. Future studies may explore the processes which account for the resolution of diarrhea despite continued bacterial presence within the intestine.

The work herein revealed unexpectedly that the localizations of Peru-NT and Peru-NTΔflaABCDE within the intestine were indistinguishable. Both strains colonized throughout the small intestine, including deep within the crypts, and were found in close apposition to the intestinal epithelium. Thus, V. cholerae does not appear dependent upon flagellar-based motility for spread within intestinal sites in this model. Flagella-independent motility has been observed for V. cholerae, although the precise mechanism underlying this process has not been identified. Brown & Hase 183 J. Bacteriol. 3784-90 (2001); Liu et al., 105 PNAS 9769-74 (2008).

The present invention thus provides for a straightforward approach to create genetically defined live-attenuated V. cholerae vaccine strains. Deletion of the two loci encoding the V. cholerae flagellins should render ctxA mutant strains non-reactogenic, while not impairing their ability to colonize the host. Furthermore, based on the existing clinical data from trials of Peru-15, which does not produce flagellins, these potent activators of innate immunity are not required to generate protective immunity against V. cholerae. Field trials of Peru-15 have yielded promising data. Qadri et al., 25 Vaccine 231-38 (2007); Qadri et al., J. Infect. Dis. 573-79 (2005). Nonetheless, the genetic plasticity of V. cholerae, as illustrated by the emergence of V. cholerae O139 in 1992 (Ramamurthy et al., 341 Lancet 703-04 (1993)), will almost certainly require construction of new vaccine strains. Deletion of the genes encoding flagellin as provided herein can be a standard part of the blueprint for creation of new live-attenuated V. cholerae vaccine strains, and for those of live-attenuated vaccines against other enteric and non-enteric pathogens that produce flagellins.

Other enteric pathogens for which reactogenicity may be decreased by deleting at least a portion of one or more flagellin genes include C. jejuni, flagellins flaA, flaB, and flaC genes (see U.S. Pat. No. 7,192,725); S. enterica, flagellins fliC and fljB genes (Mortminer et al., 7 Infect. Genet. & Evol. 411-15 (2007)); Shigella, fliC genes; E. coli, fliCE genes (Tominaga et al., 76 Gene & Genet Sys. 111-20 (2001)); and Aeromonas hydrophila, flaA and flaB flagellin genes or other genes within the flg locus (Altarriba et al., 34 Microbial Path. 249-59 (2003)).

The present invention may be defined in any of the following numbered paragraphs:

1. A method of reducing the reactogenicity of a live attentuated bacterial vaccine comprising deleting at least a portion of at least one of the bacterial genes that encodes a flagellin from the bacterium genome.

2. The method of paragraph 1, wherein said bacterium is an enteric bacterium.

3. The method of paragraph 2, wherein said bacterium is a Vibrio, Escherichia coli, Campylobacter, Salmonella, Shigella, or Aeromonas.

4. The method of paragraph 3, wherein said bacterium is Vibrio cholerae.

5. The method of paragraph 3, wherein said bacterium is enterotoxigenic Escherichia coli, enterohemmorrhagic E. coli, enteroaggregative E. coli, enteroinvasive E. coli, extraintestinal E. coli, uropathogenic E. coli. Campylobacter jejuni, Vibrio parahemolyticus, Salmonella enterica, or Aeromonas hydrophila,

6. The method of paragraph 1, wherein said bacterium is a non-enteric bacterium.

7. The method of paragraph 6, wherein said bacterium is a Pseudomonas.

8. The method of paragraph 7, wherein said Pseudomonas is Pseudomonas aeroguniosa.

9. The method of paragraph 1, wherein the portion of bacterial flagellin gene deleted is the TLR-5-stimulating domain of the flagellin protein.

10. The method of paragraph 9, wherein said bacterium is Vibrio cholerae.

### EXAMPLES

### Example 1

### Culture Conditions and Deletion Mutants

The V. cholerae strains used herein are all derivatives of the El Tor clinical isolate C6706 and are listed in Table 1:

TABLE 1
V. cholerae strains used in this study.

| V. cholerae | | |
|---|---|---|
| Strains | Description | Reference or source |
| Peru-3 | C6709 Δcore, ΔattRS1, ΔrecA::htpGp-ctxB | (1) |
| Peru-15 | C6709, spontaneous non-motile mutant of Peru-3 | (2) |
| Peru-NT | C6706 ΔctxAB | E. A. Shakhnovich |
| HR82 | Peru-NT ΔflaABCDE ΔlacZ::lac-gfp | This study |
| HR68 | Peru-NT ΔmotB ΔlacZ::lac-gfp | This study |
| HR80 | Peru-NT ΔflaA ΔlacZ::lac-gfp | This study |
| HR109 | Peru-NT ΔflaACD ΔlacZ::lac-gfp | This study |
| HR99 | Peru-NT ΔflaBCDE ΔlacZ::lac-gfp | This study |
| HR125 | Peru-NT ΔflaABCDE ΔlacZ::lac-gfp pSW-flaA | This study |
| HR118 | Peru-NT ΔhlyA Δhap Δrtx ΔlacZ::lac-gfp | This study |

(1) Taylor, D. N., Killeen, K. P., Hack, D. C., Kenner, J. R., Coster, T. S., Beattie, D. T., Ezzell, J., Hyman, T., Trofa, A., Sjogren, M. H. & et al. (1994) J Infect Dis 170, 1518-23.
(2) Kenner, J. R., Coster, T. S., Taylor, D. N., Trofa, A. F., Barrera-Oro, M., Hyman, T., Adams, J. M., Beattie, D. T., Killeen, K. P., Spriggs, D. R. & et al. (1995) J Infect Dis 172, 1126-9.

In addition to the genotypes noted within the text, all strains contained a chromosomal copy of gfp, under the control of lac, integrated within lacZ. All bacterial strains were routinely grown in Luria-Bertani (LB) medium, and maintained at −80° C. in LB broth containing 20% (vol/vol) glycerol. V. cholerae strains were grown overnight at 30° C. prior to inoculation into infant rabbits. Antibiotics were used at following concentrations: streptomycin, 200 μg mL⁻¹; spectinomycin, 50 μg mL⁻¹; and carbenicillin, 50 μg mL⁻¹.

The deletion mutants were constructed in C6706 ctxAB lacZ::gfp by allelic exchange using vectors based on pCVD442. Putative mutants were confirmed by PCR analysis. Plasmid pJZ111 was used to introduce a lac::gfp fusion (gene encoding GFP) into the V. cholerae lacZ locus. A derivative of the plasmid pSW25T, which is stably maintained in V. cholerae in vitro and in vivo without selection, was used to re-introduce the intact flaA gene under the control of its native promoter into Peru-NTΔflaABCDE.

### Example 2

### Infant Rabbit Model

Infant rabbit experiments were carried out as recently described (Ritchie et al., submitted). Briefly, 3-day old infant rabbits were treated with cimetidine (50 mg kg⁻¹ IP) 3 hr prior to oro-gastric inoculation with V. cholerae strains. In all experiments, rabbits were inoculated with ˜1×10⁹ CFU of V. cholerae suspended in sodium bicarbonate solution (2.5 g in 100 mL; pH 9). The rabbits were monitored twice daily for signs of illness. Diarrhea was scored as follows: none, no fecal material evident on the perianal area, tail or hind limbs; mild, light fecal staining of the perineum or hind legs or tail; severe, fecal material consisting of unformed or liquid stool staining large portions of the perineum, hind legs, and tail (see FIGS. 1A and 1B). The rabbits were usually necropsied at three days post-inoculation, and samples collected for histologic and microscopic analyses, RNA extraction, as well as for determining the numbers of V. cholerae CFU g⁻¹ of tissue. Some rabbits were necropsied at 6 days post-inoculation. The infant rabbits were housed with the adult female for the duration of the experiments. To limit litter-specific effects, at least two independent litters were used to test each mutant.

### Example 3

### Confocal Microscopy

Intestinal tissue was prepared for confocal microscopy as previously described (Ritchie et al., submitted). Briefly, tissue segments were fixed for 2 hr in 4% paraformaldehyde (in PBS) on ice then transferred into 30% sucrose (in PBS) at 4° C. overnight. After washing in PBS, the outer surface of the tissue segments was dried on filter paper, and trimmed pieces placed in OCT compound (Electron Microscopy Sciences, PA). Each tissue block was quick-frozen, and ˜5 μm thick sections were cut and placed on glass slides for immunofluorescence processing. Residual OCT compound was removed from the tissue by washing 3× in PBS, then the slides were stained for 1 hr with Alexa Fluor 568 phalloidin (1/50; A12380, Invitrogen) and/or Alexa Fluor 633 wheat germ agglutinin (1/200; W21404, Invitrogen) at room temperature in the dark. After further washing in PBS, the slides were counterstained with 4′-6-Diamidino-2-phenylindole (DAPI) for 5 min (1 μg mL⁻¹), washed in PBS, covered in mounting media (Vector Laboratories, CA) cover-slipped, sealed with VALAP and stored at −20° C. A Zeiss LSM 510 Meta upright confocal microscope was used to examine the slides.

### Example 4

### RNA Isolation and Quantitative Real-Time PCR

Quantitative real-time PCR (QPCR) assays were performed as previously described (34). Quinones et al., 74 Infect. Immun. 927-30 (2006). Briefly, RNA was isolated from tissue sections homogenized in Trizol (Invitrogen), then treated with DNase I (Ambion) on RNeasy mini columns (QIAGEN). Specific DNA primers, which were designed using Primer Express 2 software, were used for the reverse transcription reactions (sequences are available on request). Each RT reaction mixture contained 5 μg RNA. SYBR Green PCR master mix and an ABI Prism 7000 (Applied Biosystems) were used to perform QPCR experiment. GAPDH was used as a control gene, and all genes transcript levels were normalized to GAPDH transcript levels using the ΔΔCT method as described. Livak & Schmittgen, 25 Methods 402-08 (2001).

1. A method of reducing the reactogenicity of a live attentuated bacterial vaccine comprising deleting at least a portion of at least one of the bacterial genes that encodes a flagellin from the bacterium genome.

2. The method of claim 1, wherein said bacterium is an enteric bacterium.

3. The method of claim 2, wherein said bacterium is a Vibrio, Escherichia coli, Campylobacter, Salmonella, Shigella, or Aeromonas.

4. The method of claim 3, wherein said bacterium is Vibrio cholerae.

5. The method of claim 3, wherein said bacterium is enterotoxigenic Escherichia coli, enterohemmorrhagic E. coli, enteroaggregative E. coli, enteroinvasive E. coli, extraintestinal E. coli, uropathogenic E. coli, Campylobacter jejuni, Vibrio parahemolyticus, Salmonella enterica, or Aeromonas hydrophila,

6. The method of claim 1, wherein said bacterium is a non-enteric bacterium.

7. The method of claim 6, wherein said bacterium is a Pseudomonas.

8. The method of claim 7, wherein said Pseudomonas is Pseudomonas aeroguniosa.

9. The method of claim 1, wherein the portion of bacterial flagellin gene deleted is the TLR-5-stimulating domain of the flagellin protein.

10. The method of claim 9, wherein said bacterium is Vibrio cholerae.
